(19) United States
(12) Patent Application Publication
(10) Pub. No.: US 2009/0005756 A1
(43) Pub. Date: Jan. 1, 2009

(54) Methods and Systems of Treating Strabismus

(75) Inventor: Allison M. Foster, Valencia, CA (US)

Correspondence Address: ADVANTEDGE LAW GROUP , LLC, 3301 N. UNIVERSITY AVE ., SUITE 200, PROVO, UT, 84604, US

(73) Assignee: Boston Scientific Neuromodulation Corporation, Valencia, CA (US)

(21) Appl. No.: 12/130,058

(22) Filed: May 30, 2008

Related U.S. Application Data
(60) Provisional application No. 60/947,333, filed on Jun. 29, 2007.

(51) Int. Cl. A61M 5/00; A61N 1/08

(52) U.S. Cl. 604/521; 607/59

(57) ABSTRACT

Methods and systems of treating a patient with strabismus include providing a stimulator, configuring one or more stimulation parameters to treat strabismus, programming the stimulator with the one or more stimulation parameters, generating a stimulus configured to treat strabismus with the stimulator in accordance with the one or more stimulation parameters, and applying the stimulus with the stimulator to one or more of the extraocular muscles and/or one or more cranial nerves that innervate the extraocular muscles in accordance with the one or more stimulation parameters.

### RELATED APPLICATIONS

The present application claims priority under 35 U.S.C. §119(e) to U.S. Provisional Patent Application No. 60/947,333 by Allison M. Foster, filed on Jun. 29, 2007, and entitled “Methods and Systems of Treating Strabismus,” the contents of which are hereby incorporated by reference in their entirety.

### BACKGROUND

Strabismus is a vision disorder in which the eyes are not properly aligned with each other. The disorder is commonly referred to as “wandering eye” or “cross-eyed.” In strabismus, one or both of the eyes may point in, out, up, or down. The disorder may be constant (i.e., the eyes are always misaligned) or intermittent (i.e., the eyes are misaligned only some of the time, such as under stressful conditions or when ill).

Strabismus may be congenital, or may arise later in life due to trauma, blindness in one eye, neurologic instability, or tumors encroaching on one or more of the cranial nerves that control the extraocular muscles. Without corrective treatment, strabismus can lead to significant visual problems such as, but not limited to, diplopia (double vision), loss of depth perception, and perceptual suppression of the input from one eye. Strabismus is also a major cause of amblyopia (commonly referred to as “lazy eye”), wherein sensory input from the deviating eye is ignored despite the capacity for normal vision in that eye.

The appearance of strabismus is also a cosmetic problem for many patients. Up to 85 percent of strabismus patients report having problems with work, school and sports because of the cosmetic effects of strabismus.

Treatment options for strabismus are limited and have mixed results. Typical treatments include prism glasses, eye patches over the “good” eye, botulinum toxin (Botox) injections to relax tightened extraocular muscles, and extraocular muscle surgery. While treatment in early childhood often results in the best outcomes, the prosthetic devices used for treatment may exacerbate the social problems children with strabismus often face.

Surgical treatment of strabismus includes cutting the insertion points of the affected extraocular muscles and reattaching them at a location on the eyeball that will correct the misalignment. This surgery often results in pain, scarring, impaired eye movements, and continued misalignment. Moreover, the success rate of extraocular muscle surgery for strabismus is typically less than 50 percent.

### SUMMARY

Methods of treating a patient with strabismus include providing a stimulator, configuring one or more stimulation parameters to treat strabismus, programming the stimulator with the one or more stimulation parameters, generating a stimulus configured to treat strabismus with the stimulator in accordance with the one or more stimulation parameters, and applying the stimulus with the stimulator to one or more of the extraocular muscles and/or one or more cranial nerves that innervate the extraocular muscles in accordance with the one or more stimulation parameters.

Systems for treating a patient with strabismus include a stimulator configured to generate at least one stimulus in accordance with one or more stimulation parameters adjusted to treat strabismus, a programmable memory unit in communication with the stimulator and programmed to store the one or more stimulation parameters to at least partially define the stimulus such that the stimulus is configured to treat the strabismus, and means, operably connected to the stimulator, for applying the stimulus to one or more of the extraocular muscles and/or one or more cranial nerves that innervate the extraocular muscles.

### BRIEF DESCRIPTION OF THE DRAWINGS

The accompanying drawings illustrate various embodiments of the principles described herein and are a part of the specification. The illustrated embodiments are merely examples and do not limit the scope of the disclosure.

FIG. 1A is a front view of the left eye and shows a number of extraocular muscles that control the eye.

FIG. 1B is an anterior view of the eye, various extraocular muscles that control the eye, and various cranial nerves that innervate the extraocular muscles.

FIG. 1C is a side view of the eye and various cranial nerves that innervate the extraocular muscles.

FIG. 2 illustrates an exemplary implantable stimulator according to principles described herein.

FIG. 3 illustrates an exemplary microstimulator according to principles described herein.

FIG. 4A shows an example of a microstimulator with one or more leads coupled thereto according to principles described herein.

FIG. 4B shows an example of a microstimulator with a plurality of electrodes disposed on an outer surface thereof according to principles described herein.

FIG. 4C shows the exemplary microstimulator of FIG. 4B coupled to a lead having a number of electrodes disposed thereon.

FIG. 5 depicts a number of stimulators configured to communicate with each other and/or with one or more external devices according to principles described herein.

FIG. 6 shows a flow chart of an exemplary method of treating strabismus according to principles described herein.

FIGS. 7-8 illustrate exemplary configurations wherein one or more electrodes coupled to an implantable stimulator are in communication with one or more stimulation sites within a patient according to principles described herein.

Throughout the drawings, identical reference numbers designate similar, but not necessarily identical, elements.

### DETAILED DESCRIPTION

Methods and systems for treating a patient with strabismus are described herein. A stimulator is configured to apply at least one stimulus to one or more extraocular muscles and/or to one or more cranial nerves that innervate the extraocular muscles in accordance with one or more stimulation parameters. The stimulus is configured to treat strabismus and may include electrical stimulation and/or drug stimulation. As used herein, “treating” strabismus refers to any amelioration or prevention of one or more causes, symptoms, and/or sequalae of strabismus.

A number of advantages are associated with the systems and methods described herein. For example, the techniques used to implant the stimulator are minimally invasive and carry a low risk of external scarring. The procedures described herein for treating strabismus are reversible in that the implanted stimulator may be turned off and/or removed at any time. Moreover, adjustments to the stimulation parameters may be made throughout the treatment period by reprogramming the implanted stimulator via, for example, a transcutaneous communication link.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

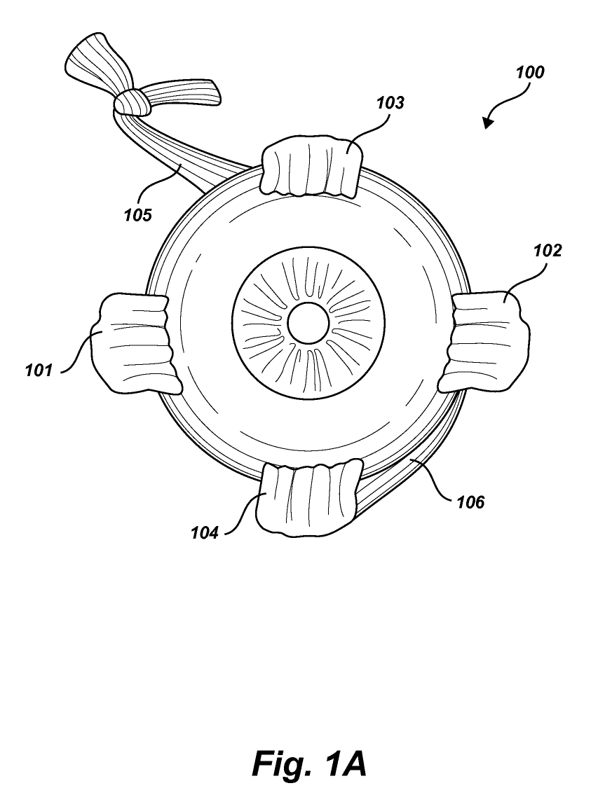

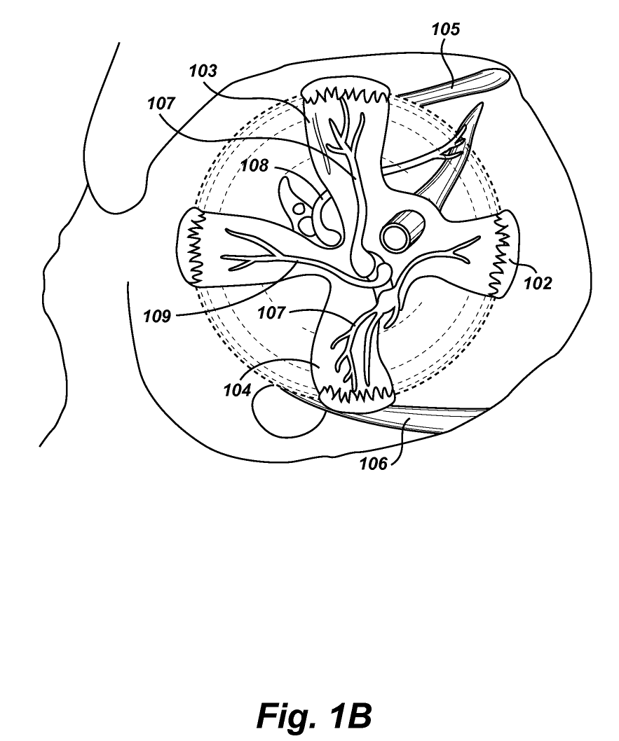

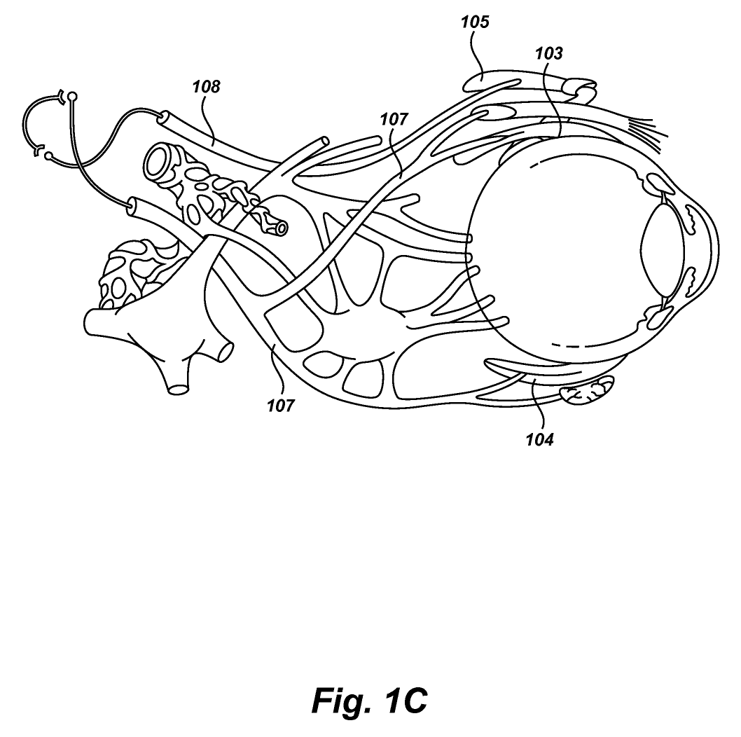

To facilitate an understanding of the systems and methods described herein, a brief overview of the etiology of strabismus will be given in connection with FIGS. 1A-1C. FIG. 1A is a front view of the left eye 100 and shows a number of extraocular muscles that control the eye 100. Static eye position and eye movements are controlled by the extraocular muscles, and strabismus is caused by their disordered control or anatomy.

As shown in FIG. 1A, there are six extraocular muscles per eye: the medial rectus 101, lateral rectus 102, superior rectus 103, inferior rectus 104, superior oblique 105, and inferior oblique 106. The medial rectus 101 moves the eye inward, towards the nose. The lateral rectus 102 moves the eye outward, away from the nose. The superior rectus 103 moves the eye upward. The inferior rectus 104 moves the eye downward. The superior oblique 105 rotates the eye so that the top of the eye moves towards the nose. The inferior oblique 106 rotates the eye so that the top of the eye moves away from the nose.

As shown in FIG. 1A, the extraocular muscles are arranged in antagonistic pairs to move the eye in opposing directions. For example, the medial and lateral rectus muscles 101 and 102 control horizontal movements and the superior and inferior oblique muscles 105 and 106 control up-and-down movements.

The extraocular muscles shown in FIG. 1A are controlled by three cranial nerves that arise directly from oculomotor nuclei within the medial longitudinal fasciculus of the brain. These cranial nerves are illustrated in the various views of the eye shown in FIGS. 1B and 1C and include the oculomotor nerve (cranial nerve 111) 107, the trochlear nerve (cranial nerve IV) 108, and the abducens nerve (cranial nerve VI) 109. The oculomotor nerve 107 innervates the medial rectus 101, the superior rectus 103, the inferior rectus 104, and the inferior oblique muscles 106. The trochlear nerve 108 innervates the superior oblique muscle 105 and the abducens nerve 109 innervates the lateral rectus muscle 102. A pathway known as the medial longitudinal fasciculus interconnects the three cranial nuclei and allows for integration of movements across all of the extraocular muscles.

When one or more of the extraocular muscles do not function properly, one or more types or of strabismus may result. These types of strabismus include, but are not limited to, esotropia, exotropia, hypertropia, hypotropia, nystagmus, and amblyopia. Esotropia refers to a condition wherein one or both of the eyes turn inward. Exotropia is a form of strabismus wherein the eyes are deviated outward. Hypertropia is a condition wherein the visual axis of one eye is higher than the other fixating eye. Hypotropia is a condition wherein the visual axis of one eye is lower than the other fixating eye. Nystagmus refers to rapid involuntary oscillation of the eyes. Amblyopia, as described previously, refers to a condition wherein sensory input from a deviating eye is ignored despite the capacity for normal vision in that eye.

It is believed that applying a stimulus to one or more of the extraocular muscles and/or one or more of the cranial nerves that innervate the extraocular muscles may be useful in treating strabismus. For ease of explanation, the term “stimulation site” will be used herein to refer generally to one or more of the extraocular muscles and/or one or more of the cranial nerves that innervate the extraocular muscles. Hence, references that are made to “stimulating a stimulation site” and variations thereof refer to applying a stimulus to one or more of the medial rectus, the lateral rectus, the superior rectus, the inferior rectus, the superior oblique, the inferior oblique, the oculomotor nerve, the trochlear nerve, and/or the abducens nerve.

For example, the stimulus may be configured to induce contraction and/or relaxation of one or more of the extraocular muscles, thereby facilitating a change in the alignment of one or more of the eyes. To illustrate, if a particular patient is experiencing esotropia (where one or both of the eyes turn inward), the stimulation parameters may be configured such that the stimulus relaxes the medial rectus muscle 101 and contracts the lateral rectus 102. In this manner, the eye may be rotated back towards a desired alignment. Likewise, if a particular patient is experiencing hypertropia (where the visual axis of one of the eyes is higher than the other fixating eye), the stimulation parameters may be configured such that the stimulus relaxes the superior rectus 103 and contracts the inferior rectus 104. It will be recognized that the preceding examples are merely illustrative and that the stimulus may be configured to treat these and other types of strabismus in any other suitable manner.

Consequently, a stimulator may be implanted within a patient to deliver a stimulus to one or more of the extraocular muscles and/or one or more of the cranial nerves that innervate the extraocular muscles to treat strabismus. The stimulus may include an electrical stimulation current and/or the infusion of one or more therapeutic drugs at the stimulation site.

As used herein, and in the appended claims, the term “stimulator” will be used broadly to refer to any device that delivers a stimulus to a stimulation site to treat strabismus. Thus, the term “stimulator” includes, but is not limited to, a microstimulator, implantable pulse generator (IPG), spinal cord stimulator (SCS), external trial stimulator, system control unit, deep brain stimulator, drug pump, or similar device.

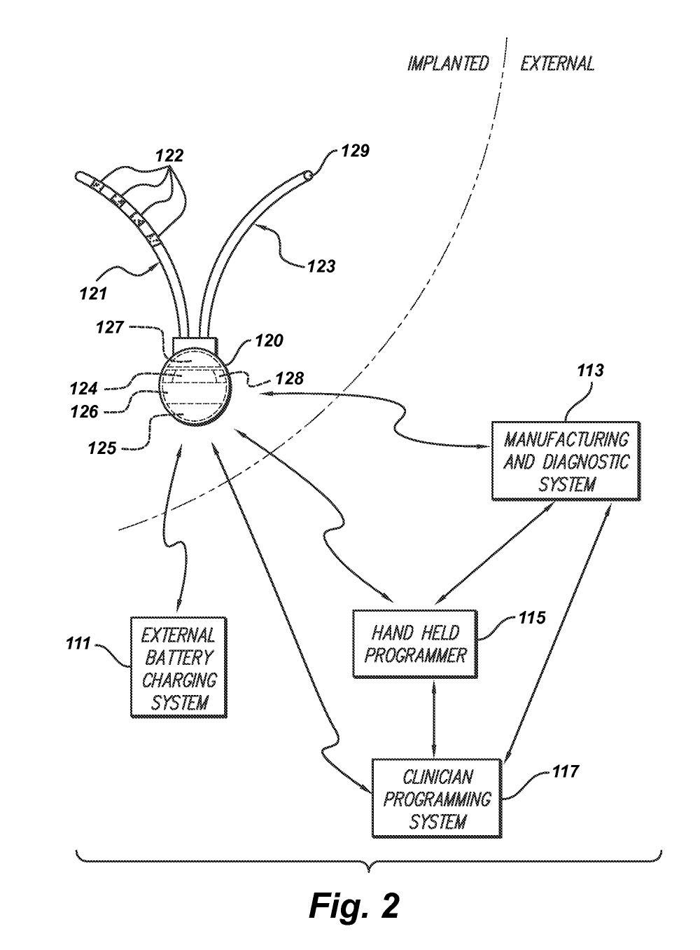

A more detailed description of an exemplary stimulator and its operation will now be given in connection with FIG. 2. FIG. 2 illustrates an exemplary stimulator 120 that may be used to apply a stimulus to a stimulation site within a patient, e.g., an electrical stimulation of the stimulation site, an infusion of one or more drugs at the stimulation site, or both. The electrical stimulation function of the stimulator 120 will be described first, followed by an explanation of the possible drug delivery function of the stimulator 120. It will be understood, however, that the stimulator 120 may be configured to provide only electrical stimulation, only drug stimulation, both types of stimulation, or any other type of stimulation as best suits a particular patient.

The exemplary stimulator 120 shown in FIG. 2 is configured to provide electrical stimulation to one or more stimulation sites within a patient and may include at least one lead 121 coupled thereto. In some examples, the at least one lead 121 includes a number of electrodes 122 through which electrical stimulation current may be applied to a stimulation site. It will be recognized that the at least one lead 121 may include any number of electrodes 122 arranged in any configuration as best serves a particular application. In some alternative examples, as will be described in more detail below, the stimulator 120 is leadless.

As illustrated in FIG. 2, the stimulator 120 includes a number of components. It will be recognized that the stimulator 120 may include additional and/or alternative components as best serves a particular application. A power source 125 is configured to output voltage used to supply the various components within the stimulator 120 with power and/or to generate the power used for electrical stimulation. The power source 125 may include a primary battery, a rechargeable battery (e.g., a lithium-ion battery), a super capacitor, a nuclear battery, a mechanical resonator, an infrared collector (receiving, e.g., infrared energy through the skin), a thermally-powered energy source (where, e.g., memory-shaped alloys exposed to a minimal temperature difference generate power), a flexural powered energy source (where a flexible section subject to flexural forces is part of the stimulator), a bioenergy power source (where a chemical reaction provides an energy source), a fuel cell, a bioelectrical cell (where two or more electrodes use tissue-generated potentials and currents to capture energy and convert it to useable power), an osmotic pressure pump (where mechanical energy is generated due to fluid ingress), or the like.

In some examples, the power source 125 may be recharged using an external charging system. One type of rechargeable power supply that may be used is described in U.S. Pat. No. 6,596,439, which is incorporated herein by reference in its entirety. Other battery construction techniques that may be used to make the power source 125 include those shown, e.g., in U.S. Pat. Nos. 6,280,873; 6,458,171; 6,605,383; and 6,607,843, all of which are incorporated herein by reference in their respective entireties.

The stimulator 120 may also include a coil 128 configured to receive and/or emit a magnetic field (also referred to as a radio frequency (RF) field) that is used to communicate with, or receive power from, one or more external devices. Such communication and/or power transfer may include, but is not limited to, transcutaneously receiving data from the external device, transmitting data to the external device, and/or receiving power used to recharge the power source 125.

For example, an external battery charging system (EBCS) 111 may be provided to generate power that is used to recharge the power source 125 via any suitable communication link. Additional external devices including, but not limited to, a hand held programmer (HHP) 115, a clinician programming system (CPS) 117, and/or a manufacturing and diagnostic system (MDS) 113 may also be provided and configured to activate, deactivate, program, and/or test the stimulator 120 via one or more communication links. It will be recognized that the communication links shown in FIG. 2 may each include any type of link used to transmit data or energy, such as, but not limited to, an RF link, an infrared (IR) link, an optical link, a thermal link, or any other energy-coupling link.

Additionally, if multiple external devices are used in the treatment of a patient, there may be communication among those external devices, as well as with the implanted stimulator 120. It will be recognized that any suitable communication link may be used among the various devices illustrated.

The external devices shown in FIG. 2 are merely illustrative of the many different external devices that may be used in connection with the stimulator 120. Furthermore, it will be recognized that the functions performed by any two or more of the external devices shown in FIG. 2 may be performed by a single external device.

The stimulator 120 may also include electrical circuitry 124 configured to generate the electrical stimulation current that is delivered to a stimulation site via one or more of the electrodes 122. For example, the electrical circuitry 124 may include one or more processors, capacitors, integrated circuits, resistors, coils, and/or any other component configured to generate electrical stimulation current.

Additionally, the exemplary stimulator 120 shown in FIG. 2 may be configured to provide drug stimulation to a patient by applying one or more drugs at a stimulation site within the patient. To this end, a pump 127 may also be included within the stimulator 120. The pump 127 is configured to store and dispense one or more drugs, for example, through a catheter 123. The catheter 123 is coupled at a proximal end to the stimulator 120 and may have an infusion outlet 129 for infusing dosages of the one or more drugs at the stimulation site. In some embodiments, the stimulator 120 may include multiple catheters 123 and/or pumps 127 for storing and infusing dosages of the one or more drugs at the stimulation site.

The one or more drugs that may be applied to a stimulation site to treat strabismus may have an excitatory effect on the stimulation site (e.g., induce contractions of one or more of the extraocular muscles). Additionally or alternatively, the one or more drugs may have an inhibitory effect on the stimulation site (e.g., induce relaxation of one or more of the extraocular muscles). Exemplary excitatory drugs that may be applied to a stimulation site to treat strabismus include, but are not limited to, at least one or more of the following: an excitatory neurotransmitter (e.g., glutamate, dopamine, norepinephrine, epinephrine, acetylcholine, serotonin); an excitatory neurotransmitter agonist (e.g., glutamate receptor agonist, L-aspartic acid, N-methyl-D-aspartic acid (NMDA), bethanechol, norepinephrine); an inhibitory neurotransmitter antagonist(s) (e.g., bicuculline); an agent that increases the level of an excitatory neurotransmitter (e.g., edrophonium, Mestinon); and/or an agent that decreases the level of an inhibitory neurotransmitter (e.g., bicuculline).

Exemplary inhibitory drugs that may be applied to a stimulation site to treat strabismus include, but are not limited to, at least one or more of the following: an inhibitory neurotransmitter(s) (e.g., gamma-aminobutyric acid, a.k.a. GABA, dopamine, glycine); an agonist of an inhibitory neurotransmitter (e.g., a GABA receptor agonist such as midazolam or clondine, muscimol); an excitatory neurotransmitter antagonist(s) (e.g. prazosin, metoprolol, atropine, benztropine); an agent that increases the level of an inhibitory neurotransmitter; an agent that decreases the level of an excitatory neurotransmitter (e.g., acetylcholinesterase, Group II metabotropic glutamate receptor (mGluR) agonists such as DCG-IV); a local anesthetic agent (e.g., lidocaine); and/or an analgesic medication. It will be understood that some of these drugs, such as dopamine, may act as excitatory neurotransmitters in some stimulation sites and circumstances, and as inhibitory neurotransmitters in other stimulation sites and circumstances.

Additional or alternative drugs that may be applied to a stimulation site to treat strabismus include, but are not limited to, neurotrophic factors (e.g., brain derived neurotrophic factors (BDNF) and glial cell line derived neurotrophic factors (GDNF)), steroids, antibiotics, anticonvulsants, antidepressants, and gangliosides. These compounds have been shown to increase efficacy of drug infusion, reduce fibrosis, and/or prevent infection.

Any of the drugs listed above, alone or in combination, or other drugs or combinations of drugs developed or shown to treat strabismus may be applied to the stimulation site. In some embodiments, the one or more drugs are infused chronically into the stimulation site. Additionally or alternatively, the one or more drugs may be infused acutely into the stimulation site in response to a biological signal or a sensed need for the one or more drugs.

The stimulator 120 may also include a programmable memory unit 126 configured to store one or more stimulation parameters. The stimulation parameters may include, but are not limited to, electrical stimulation parameters, drug stimulation parameters, and other types of stimulation parameters. The programmable memory unit 126 allows a patient, clinician, or other user of the stimulator 120 to adjust the stimulation parameters such that the stimulation applied by the stimulator 120 is safe and efficacious for treatment of a particular patient. The programmable memory unit 126 may include any type of memory unit such as, but not limited to, random access memory (RAM), static RAM (SRAM), a hard drive, or the like.

The electrical stimulation parameters may control various parameters of the stimulation current applied to a stimulation site including, but not limited to, the frequency, pulse width, amplitude, waveform (e.g., square or sinusoidal), electrode configuration (i.e., anode-cathode assignment), burst pattern (e.g., continuous or intermittent), duty cycle or burst repeat interval, ramp on time, and ramp off time. The drug stimulation parameters may control various parameters including, but not limited to, the amount of drugs infused at the stimulation site, the rate of drug infusion, and the frequency of drug infusion. For example, the drug stimulation parameters may cause the drug infusion rate to be intermittent, continuous, or bolus.

Specific stimulation parameters may have different effects on different types, causes, or symptoms of strabismus. Thus, in some examples, the stimulation parameters may be adjusted at any time throughout the treatment course as best serves the particular patient being treated. It will be recognized that any of the characteristics of the stimulation current, including, but not limited to, the pulse shape, amplitude, pulse width, frequency, burst pattern (e.g., continuous or intermittent), duty cycle or burst repeat interval, ramp on time, and ramp off time may be adjusted throughout the course of treatment as best serves a particular application.

To illustrate, a baseline set of stimulation parameters may initially be set to begin treatment of strabismus. These baseline values may be adjusted throughout the course of treatment in response to patient feedback or sensed indicators of strabismus. Additionally or alternatively, the patient and/or clinician may adjust the stimulation parameters at any time to prevent accommodation, collateral stimulation, and/or ineffectiveness.

An exemplary baseline set of stimulation parameters that may be used to initially define stimulation current that is used to treat strabismus includes, but is not limited to the stimulation parameters shown in Table 1. It will be recognized that the baseline set of stimulation parameters shown in Table 1 may vary depending on the particular patient being treated and that additional or alternative stimulation parameters may be defined.

TABLE 1
Exemplary Baseline Stimulation Parameters

| | |
|---|---|
| Pulse width | 10 microseconds (μsec) |
| Frequency | 45-125 Hertz (Hz) to relax an |
| | extraocular muscle or 125-350 Hz |
| | to contract an |
| | extraocular muscle |
| Burst pattern | Continuous |
| Amplitude | 0.1 milliamps (mA) |

Hence, as shown in Table 1, a continuous stimulation current having a pulse width of 10 μsec and an amplitude of 0.1 mA may be initially applied to one or more of the extraocular muscles and/or one of the cranial nerves that innervate the extraocular muscles in order to treat strabismus. The pulse width and amplitude may initially have relatively small values so as to avoid muscle spasms, nerve damage, or discomfort.

As shown in Table 1, the frequency of the stimulation current depends on whether it is desirable to relax or contract the target extraocular muscle. In some patients, the eye will be stationary or static at about 125 Hz. It will be recognized that this static frequency may vary depending on the particular patient. Hence, if it is desirable to relax the target extraocular muscle (and thereby cause the eye to point away from the muscle), the frequency may be set to initially have a value less than the static frequency (e.g., between 45 and 125 Hz). If it is desirable to contract the target extraocular muscle (and thereby cause the eye to point towards the muscle), the frequency may be set to initially have a value greater than the static frequency (e.g., between 125 and 350 Hz). It will be recognized that the frequency ranges listed herein are merely exemplary and that they may be adjusted as best serves a particular patient.

In some examples, these baseline parameters may be determined in the initial fitting session and may depend on the electrode placement (e.g., how proximal they are to the stimulation site), local impedance (which may be affected by scar tissue, etc.), and patient variability. The clinician or other programmer may make subtle, iterative adjustments to any of the stimulation parameters in response to realtime feedback from the patient.

After a predetermined length of time (e.g., a week, a month, or multiple months) of treatment or as the need may arise, the patient may be evaluated to determine whether the stimulation parameters need to be adjusted and/or whether the additional stimulation is needed in order to treat strabismus. In some examples, if the patient no longer exhibits any symptoms of strabismus, the stimulation may be terminated. Alternatively, if it is determined that the patient needs further treatment, the stimulation may continue in accordance with the same set of stimulation parameters or in accordance with a newly defined set of stimulation parameters. For example, the stimulation parameters may be adjusted from the exemplary baseline stimulation parameters described previously in connection with Table 1 to have the exemplary values within the ranges shown in Table 2:

TABLE 2
Exemplary Adjusted Stimulation Parameters

| | |
|---|---|
| Pulse width | 5 μsec |
| Frequency | 45-125 Hertz (Hz) to relax an |
| | extraocular muscle or 125-350 Hz |
| | to contract an |
| | extraocular muscle |
| Burst pattern | Continuous |
| Amplitude | 0.2 mA |

As shown in Table 2, the pulse width, frequency, and/or amplitude may be adjusted so that the stimulation current more effectively treats strabismus. For example, the pulse width and amplitude may be adjusted to more suitable values (e.g., 5 μsec and 0.2 mA). It will be recognized that the values shown in Table 2 are merely illustrative and that they may vary as may serve a particular application. It will also be recognized that any other stimulation parameter (e.g., one or more of the drug stimulation parameters) may additionally or alternatively be adjusted in order to more effectively treat strabismus.

The stimulator 120 of FIG. 2 is illustrative of many types of stimulators that may be used in accordance with the systems and methods described herein. For example, the stimulator 120 may include an implantable pulse generator (IPG), a spinal cord stimulator (SCS), a deep brain stimulator, a drug pump, or any other type of implantable device configured to deliver a stimulus to a stimulation site within a patient. Exemplary IPGs suitable for use as described herein include, but are not limited to, those disclosed in U.S. Pat. Nos. 6,381,496, 6,553,263; and 6,760,626. Exemplary spinal cord stimulators suitable for use as described herein include, but are not limited to, those disclosed in U.S. Pat. Nos. 5,501,703; 6,487,446; and 6,516,227. Exemplary deep brain stimulators suitable for use as described herein include, but are not limited to, those disclosed in U.S. Pat. Nos. 5,938,688; 6,016,449; and 6,539,263. All of these listed patents are incorporated herein by reference in their respective entireties.

The stimulator 120 of FIG. 2 may alternatively include a microstimulator. Various details associated with the manufacture, operation, and use of implantable microstimulators are disclosed in U.S. Pat. Nos. 5,193,539; 5,193,540; 5,312,439; 6,185,452; 6,164,284; 6,208,894; and 6,051,017. All of these listed patents are incorporated herein by reference in their respective entireties.

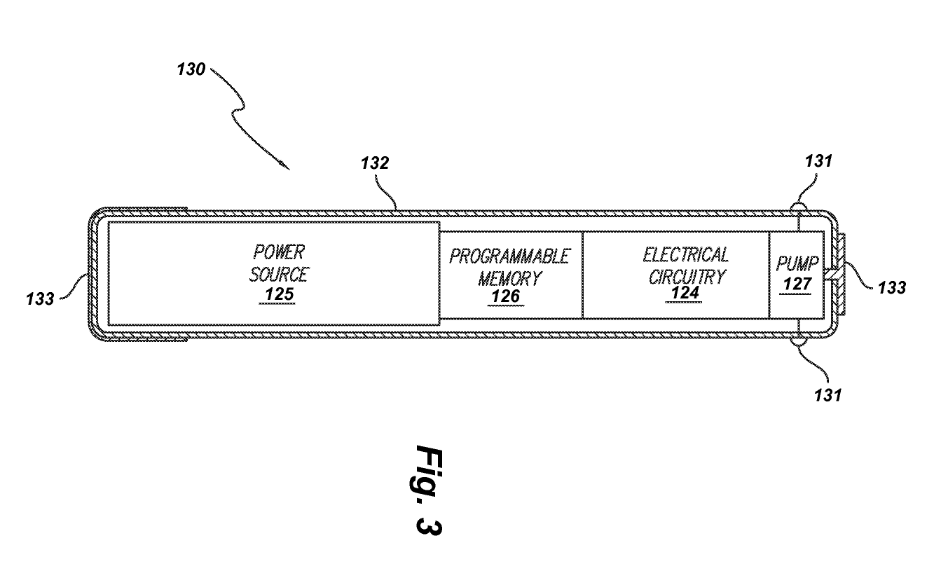

FIG. 3 illustrates an exemplary microstimulator 130 that may be used as the stimulator 120 described herein. Other configurations of the microstimulator 130 are possible, as shown in the above-referenced patents and as described further below.

As shown in FIG. 3, the microstimulator 130 may include the power source 125, the programmable memory 126, the electrical circuitry 124, and the pump 127 described in connection with FIG. 2. These components are housed within a capsule 132. The capsule 132 may be a thin, elongated cylinder or any other shape as best serves a particular application. The shape of the capsule 132 may be determined by the structure of the desired stimulation site and the method of implantation. In some examples, the microstimulator 130 may include two or more leadless electrodes 133 disposed on the outer surface thereof.

The external surfaces of the microstimulator 130 may advantageously be composed of biocompatible materials. For example, the capsule 132 may be made of glass, ceramic, metal, or any other material that provides a hermetic package that will exclude water vapor but permit passage of electromagnetic fields used to transmit data and/or power. The electrodes 133 may be made of a noble or refractory metal or compound, such as platinum, iridium, tantalum, titanium, titanium nitride, niobium or alloys of any of these, in order to avoid corrosion or electrolysis which could damage the surrounding tissues and the device.

The microstimulator 130 may also include one or more infusion outlets 131 configured to dispense one or more drugs directly at a stimulation site. Alternatively, one or more catheters may be coupled to the infusion outlets 131 to deliver the drug therapy to a treatment site some distance from the body of the microstimulator 130.

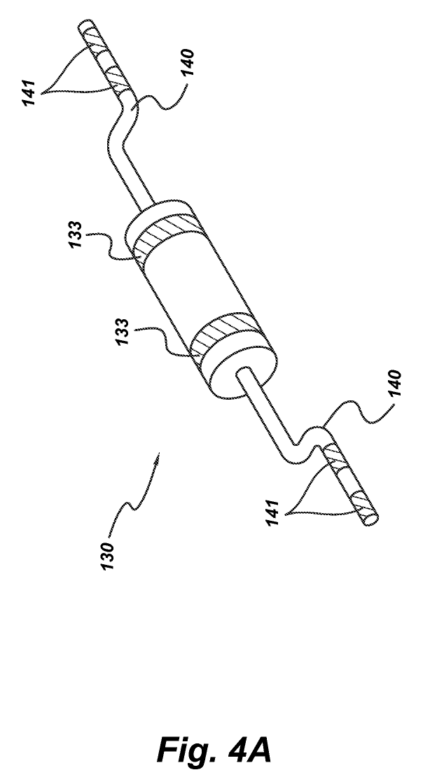

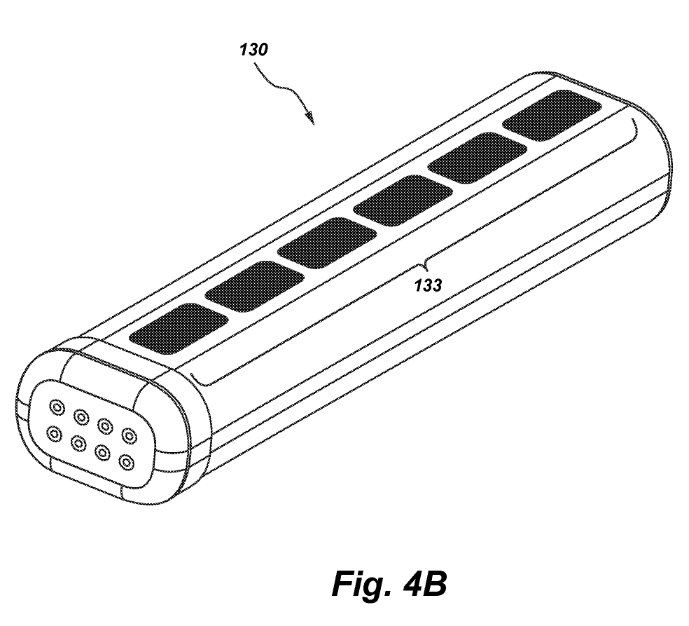

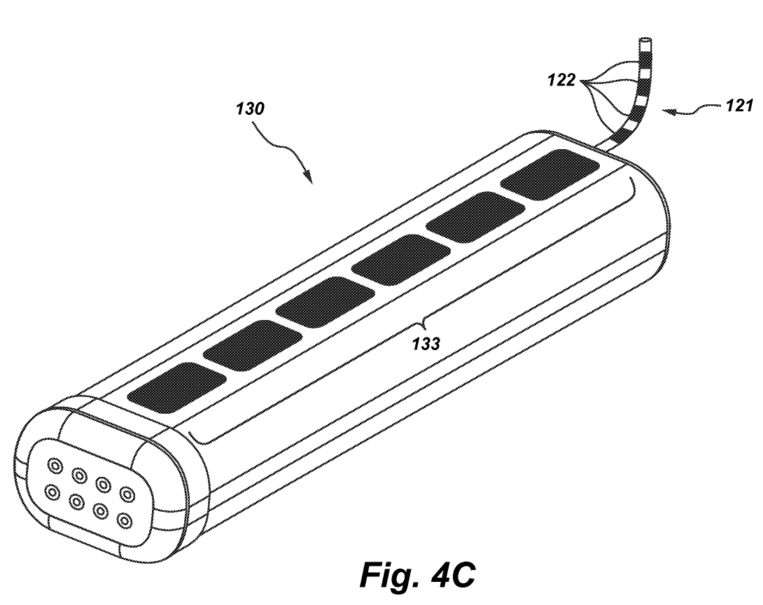

FIGS. 4A-4C show alternative configurations of a microstimulator 130. It will be recognized that the alternative configurations shown in FIGS. 4A-4C are merely illustrative of the many possible configurations of a microstimulator 130. For example, FIG. 4A shows an example of a microstimulator 130 with one or more leads 140 coupled thereto. As shown in FIG. 4A, each of the leads 140 may include one or more electrodes 141 disposed thereon. The microstimulator 130 of FIG. 4A may additionally or alternatively include one or more leadless electrodes 133 disposed on the outer surface thereof.

FIG. 4B illustrates an exemplary microstimulator 130 with a plurality of electrodes 133 disposed on an outer surface thereof. In some examples, any number of electrodes 133 may be disposed on the outer surface of the microstimulator 130. In some alternative examples, as shown in FIG. 4C, the microstimulator 130 may be coupled to a lead 121 having a number of electrodes 122 disposed thereon. Each of the electrodes 133 and 122 may be selectively configured to serve as an anode or as a cathode.

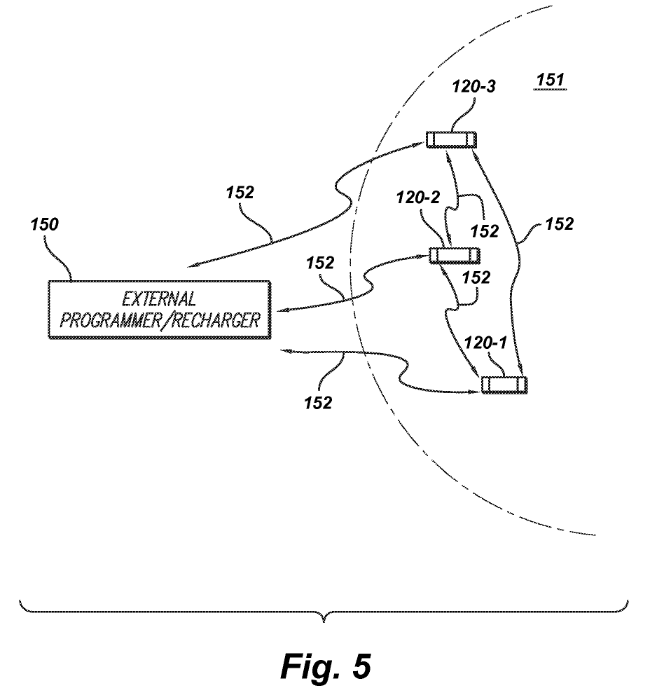

In some examples, the stimulator 120 of FIG. 2 may be configured to operate independently. Alternatively, as shown in FIG. 5, the stimulator 120 may be configured to operate in a coordinated manner with one or more additional stimulators, other implanted devices, or other devices external to the patient's body. FIG. 5 illustrates an exemplary configuration wherein a first stimulator 120-1 implanted within the patient 151 provides a stimulus to a first location, a second stimulator 120-2 provides a stimulus to a second location, and a third stimulator 120-3 provides a stimulus to a third location. In some examples, one or more external devices 150 may be configured to control the operation of each of the implanted devices 120. In some embodiments, an implanted device, e.g., stimulator 120-1, may control, or operate under the control of, another implanted device(s), e.g., stimulator 120-2 and/or stimulator 120-3. Control lines 152 have been drawn in FIG. 5 to illustrate that the external device 150 may communicate or provide power to any of the implanted devices 120 and that each of the various implanted devices 120 may communicate with and, in some instances, control any of the other implanted devices.

As a further example of multiple stimulators 120 operating in a coordinated manner, the first and second stimulators 120-1 and 120-2 of FIG. 5 may be configured to sense various indicators of the symptoms or causes of strabismus and transmit the measured information to the third stimulator 120-3. The third stimulator 120-3 may then use the measured information to adjust its stimulation parameters and apply stimulation to a stimulation site accordingly. The various implanted stimulators may, in any combination, sense indicators of strabismus, communicate or receive data regarding such indicators, and adjust stimulation parameters accordingly.

In order to determine the strength and/or duration of electrical stimulation and/or amount and/or type(s) of stimulating drug(s) required to most effectively treat strabismus, various indicators of strabismus and/or a patient's response to treatment may be sensed or measured. The stimulator 120 may then adjust the stimulation parameters (e.g., in a closed loop manner) in response to one or more of the sensed indicators. Exemplary indicators include, but are not limited to, electrical activity of the brain (e.g., EEG), neurotransmitter levels, patient input, ocular motility test results, and/or other eye examination test results. In some examples, the stimulator 120 may be configured to perform one or more of the measurements. Alternatively, other sensing devices may be configured to perform the measurements and transmit the measured values to the stimulator 120.

Thus, one or more external devices may be provided to interact with the stimulator 120, and may be used to accomplish at least one or more of the following functions:

Function 1: If necessary, transmit electrical power to the stimulator 120 in order to power the stimulator 120 and/or recharge the power source 125.

Function 2: Transmit data to the stimulator 120 in order to change the stimulation parameters used by the stimulator 120.

Function 3: Receive data indicating the state of the stimulator 120 (e.g., battery level, drug level, stimulation parameters, etc.).

Additional functions may include adjusting the stimulation parameters based on information sensed by the stimulator 120 or by other sensing devices.

By way of example, an exemplary method of treating strabismus may be carried out according to the following sequence of procedures. The steps listed below may be modified, reordered, and/or added to as best serves a particular application.

1. A stimulator 120 is implanted so that its electrodes 122 and/or infusion outlet 129 are in communication with a stimulation site within a patient. As used herein and in the appended claims, the term “in communication with” refers to the stimulator 120, stimulating electrodes 122, and/or infusion outlet 129 being adjacent to, in the general vicinity of, in close proximity to, directly next to, or directly on one or more of the extraocular muscles and/or one or more of the cranial nerves that innervate the extraocular muscles.

2. One or more stimulation parameters are configured to treat strabismus.

3. The stimulator 120 is programmed with the one or more stimulation parameters configured to treat strabismus. The stimulator 120 may then generate and apply at least one stimulus to the stimulation site in accordance with the stimulation parameters. The stimulus may include electrical stimulation, drug stimulation, gene infusion, chemical stimulation, thermal stimulation, electromagnetic stimulation, mechanical stimulation, and/or any other suitable stimulation.

4. When the patient desires to invoke stimulation, the patient sends a command to the stimulator 120 (e.g., via a remote control) such that the stimulator 120 delivers the prescribed stimulation to the stimulation site. For example, the stimulation may be activated by the patient when a particular incident of strabismus is detected. The stimulator 120 may alternatively or additionally be configured to apply the stimulation to the stimulation site in accordance with one or more pre-determined stimulation parameters and/or automatically apply the stimulation in response to sensed indicators of strabismus.

5. To cease stimulation, the patient may turn off the stimulator 120 (e.g., via a remote control).

6. Periodically, the power source 125 of the stimulator 120 is recharged, if necessary, in accordance with Function 1 described above.

In other examples, the treatment administered by the stimulator 120, i.e., drug therapy and/or electrical stimulation, may be automatic and not controlled or invoked by the patient. It will be recognized that the particular stimulation methods and parameters may vary as best serves a particular application.

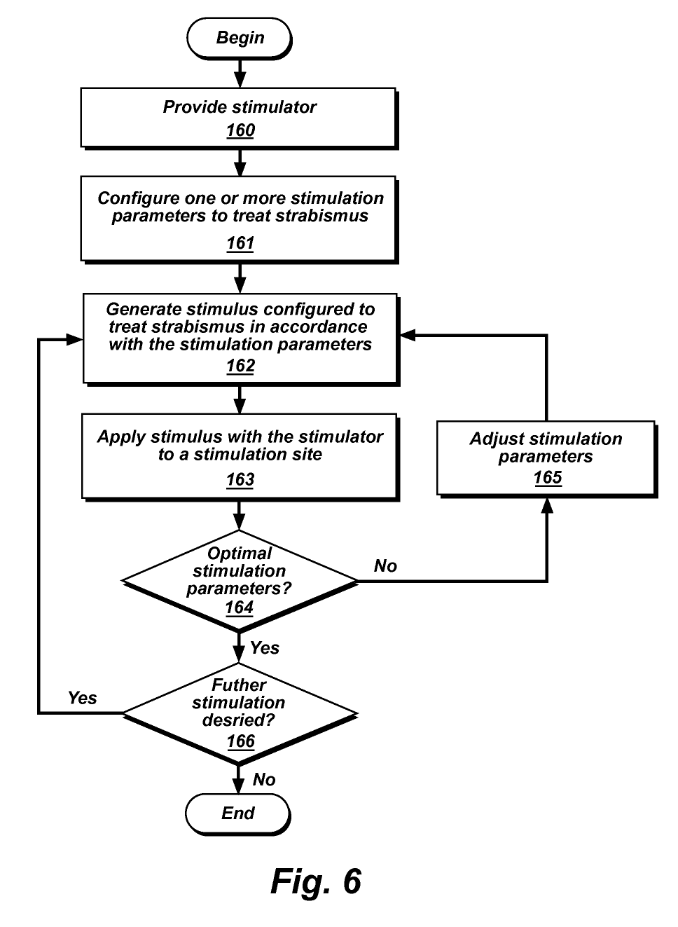

FIG. 6 shows a flow chart of an exemplary method of treating strabismus, according to the principles that have been described in more detail above. While FIG. 6 illustrates exemplary steps according to one embodiment, other embodiments may omit, add to, reorder, and/or modify any of the steps shown in FIG. 6.

In step 160, a stimulator is provided. In some examples, the stimulator may at least partially implanted within the patient. Alternatively, the stimulator may be located external to the patient.

In step 161, one or more stimulation parameters are configured to treat strabismus. The stimulation parameters may include, but are not limited to, electrical stimulation parameters, drug stimulation parameters, and/or other types of stimulation parameters.

In step 162, a stimulus is generated that is configured to treat strabismus in accordance with the stimulation parameters.

In step 163, the stimulus is applied with the stimulator to a stimulation site. The stimulation site may include, for example, an extraocular muscle and/or a cranial nerve innervating an extraocular muscle.

In step 164, it is determined whether the stimulation parameters are optimal. To this end, one or more indicators related to strabismus may be sensed.

In step 165, the stimulation parameters may be adjusted if the stimulation parameters are determined to be sub-optimal in step 164. The stimulation parameters may be adjusted in any of the ways described herein. Steps 162 through 164 may then be repeated.

The stimulus may be applied to the stimulation site until it is determined that further stimulation is not desired (No, step 166).

The stimulator 120 may be implanted within a patient using any suitable surgical procedure such as, but not limited to, small incision, open placement, laparoscopy, or endoscopy. Exemplary methods of implanting a microstimulator, for example, are described in U.S. Pat. Nos. 5,193,539; 5,193,540; 5,312,439; 6,185,452; 6,164,284; 6,208,894; and 6,051,017. Exemplary methods of implanting an SCS, for example, are described in U.S. Pat. Nos. 5,501,703; 6,487,446; and 6,516,227. Exemplary methods of implanting a deep brain stimulator, for example, are described in U.S. Pat. Nos. 5,938,688; 6,016,449; and 6,539,263. All of these listed patents are incorporated herein by reference in their respective entireties.

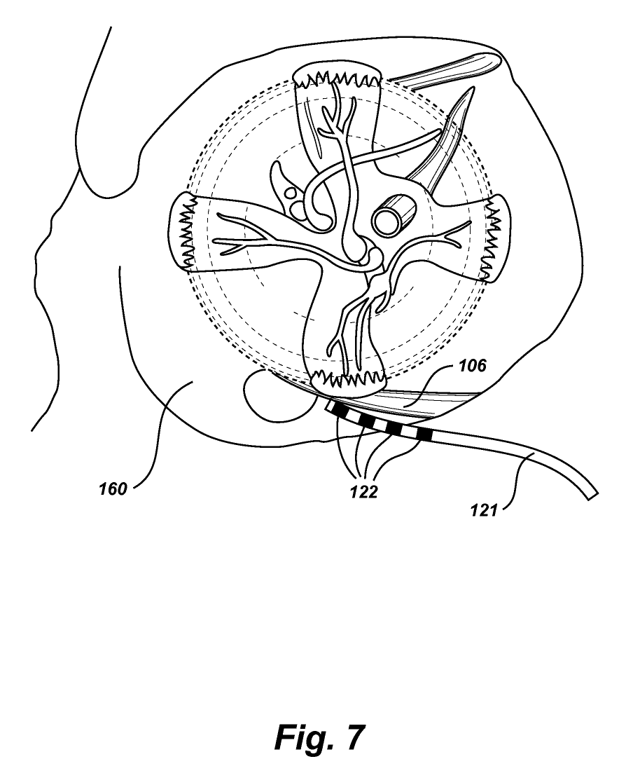

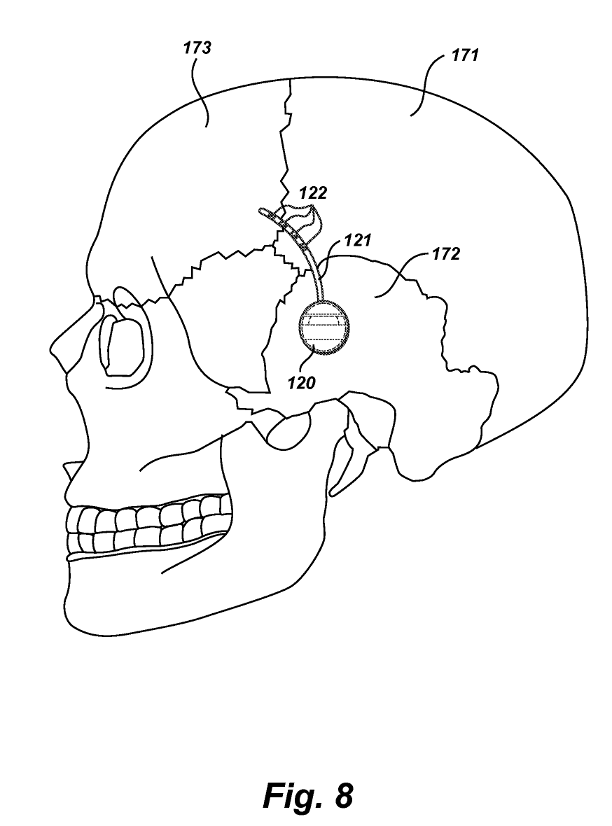

To illustrate, FIGS. 7-8 illustrate exemplary configurations wherein one or more electrodes 122 coupled to an implantable stimulator 120 are in communication with one or more of the extraocular muscles and/or one or more of the cranial nerves that innervate the extraocular muscles. The configurations shown in FIGS. 7-8 are merely illustrative of the many different implant configurations that may be used in accordance with the systems and methods described herein.

In the example of FIG. 7, the distal portion of a lead 121 having electrodes 122 disposed thereon may be placed within the orbit 160 such that the electrodes 122 are in communication with one or more of the extraocular muscles and/or one or more of the cranial nerves that innervate the extraocular muscles. For example, the electrodes 122 illustrated in FIG. 7 are in communication with the inferior oblique muscle 106.

The lead 121 shown in FIG. 7 may be coupled to a stimulator 120 that has been implanted in a more convenient location. For example, as shown in FIG. 8, the stimulator 120 may be implanted beneath the scalp, such as in a surgically-created shallow depression or opening in the skull. The surgically-created shallow depression or opening may be located in the parietal bone 171, the temporal bone 172, and/or the frontal bone 173. In some examples, the stimulator 120 is configured to conform to the profile of surrounding tissue(s) and/or bone(s). This may minimize pressure applied to the skin or scalp, which pressure may result in skin erosion or infection.

In some alternative examples, a stimulator 120 having a suitable miniature size may also be implanted within the orbit 160. It will be recognized that the lead 121 may additionally or alternatively be coupled to an external stimulating device. It will also be recognized that although only an electrode lead 122 is shown in FIGS. 7-8, a catheter 123 may additionally or alternatively be implanted for drug stimulation in a similar manner.

The preceding description has been presented only to illustrate and describe embodiments of the invention. It is not intended to be exhaustive or to limit the invention to any precise form disclosed. Many modifications and variations are possible in light of the above teaching.

What is claimed is:

1. A method of treating a patient with strabismus, comprising:
providing a stimulator;
configuring one or more stimulation parameters to treat strabismus;
programming said stimulator with said one or more stimulation parameters;
generating a stimulus configured to treat said strabismus with said stimulator in accordance with said one or more stimulation parameters; and
applying said stimulus with said stimulator to a stimulation site within said patient;
wherein said stimulation site comprises at least one or more of an extraocular muscle and a cranial nerve innervating said extraocular muscle.

2. The method claim 1, wherein said extraocular muscle comprises at least one or more of a medial rectus muscle, a lateral rectus muscle, a superior rectus muscle, an inferior rectus muscle, a superior oblique muscle, and an inferior oblique muscle.

3. The method of claim 1, wherein said cranial nerve comprises at least one or more of an oculomotor nerve, a trochlear nerve, and an abducens nerve.

4. The method of claim 1, wherein said stimulator is coupled to one or more electrodes, and wherein said stimulus comprises a stimulation current delivered via said electrodes.

5. The method of claim 1, wherein said stimulus comprises an infusion of one or more drugs at said stimulation site.

6. The method of claim 1, further comprising evaluating an effectiveness of said stimulus and adjusting said stimulation parameters in accordance with said evaluation.

7. The method of claim 1, further comprising at least partially implanting said stimulator within said patient.

8. The method of claim 1, further comprising configuring said one or more stimulation parameters such that said stimulus is configured to induce contraction of one or more of said extraocular muscles.

9. The method of claim 1, further comprising configuring said one or more stimulation parameters such that said stimulus is configured to induce relaxation of one or more of said extraocular muscles.

10. The method of claim 1, further comprising sensing at least one indicator related to said strabismus and using said at least one sensed indicator to adjust one or more of said stimulation parameters.

11. A method of treating strabismus, said method comprising:
implanting a stimulator at least partially within a patient;
configuring one or more stimulation parameters to treat strabismus;
programming said stimulator with said one or more stimulation parameters;
generating a stimulation current configured to treat said strabismus with said stimulator in accordance with said one or more stimulation parameters; and
applying said stimulation current with said implanted stimulator to a stimulation site within said patient;
wherein said stimulation site comprises at least one or more of an extraocular muscle and a cranial nerve innervating said extraocular muscle.

12. The method claim 11, wherein said extraocular muscle comprises at least one or more of a medial rectus muscle, a lateral rectus muscle, a superior rectus muscle, an inferior rectus muscle, a superior oblique muscle, and an inferior oblique muscle.

13. The method of claim 11, wherein said cranial nerve comprises at least one or more of an oculomotor nerve, a trochlear nerve, and an abducens nerve.

14. The method of claim 11, further comprising configuring said one or more stimulation parameters such that said stimulus is configured to induce contraction of one or more of said extraocular muscles.

15. The method of claim 11, further comprising configuring said one or more stimulation parameters such that said stimulus is configured to induce relaxation of one or more of said extraocular muscles.

16. The method of claim 11, further comprising sensing at least one indicator related to said strabismus and using said at least one sensed indicator to adjust one or more of said stimulation parameters.

17. A system for treating a patient with strabismus, said system comprising:
a stimulator configured to generate at least one stimulus in accordance with one or more stimulation parameters adjusted to treat strabismus;
a programmable memory unit in communication with said stimulator and programmed to store said one or more stimulation parameters to at least partially define said stimulus such that said stimulus is configured to treat said strabismus; and
means, operably connected to said stimulator, for applying said stimulus to a stimulation site within said patient;
wherein said stimulation site comprises at least one or more of an extraocular muscle and a cranial nerve innervating said extraocular muscle.

18. The system claim 17, wherein said extraocular muscle comprises at least one or more of a medial rectus muscle, a lateral rectus muscle, a superior rectus muscle, an inferior rectus muscle, a superior oblique muscle, and an inferior oblique muscle.

19. The system of claim 17, wherein said cranial nerve comprises at least one or more of an oculomotor nerve, a trochlear nerve, and an abducens nerve.

20. The system of claim 17, wherein said means for applying said at least one stimulus comprises one or more electrodes, and wherein said stimulus comprises a stimulation current delivered via said electrodes.
